(19) United States
(12) Patent Application Publication
(10) Pub. No.: US 2026/0108140 A1
(43) Pub. Date: Apr. 23, 2026

(54) DEVICES, SYSTEMS, AND METHODS FOR GUIDING AN ENDOSCOPE INSTRUMENT AND SELECTIVELY MOVING AN ATTACHABLE ULTRASOUND ASSEMBLY

(71) Applicant: EndoSound, Inc., Portland, OR (US)

(72) Inventors: Peter D. HOFFMAN, Portland, OR (US); Jeremy Andrew HAMMER, Portland, OR (US); Scott Sutherland CORBETT, III, Portland, OR (US)

(21) Appl. No.: 19/155,387

(22) PCT Filed: Feb. 12, 2024
(86) PCT No.: PCT/US2024/015362

§ 371 Date: Aug. 11, 2025

Related U.S. Application Data
(60) Provisional application No. 63/484,556, filed on Feb. 13, 2023.

(51) Int. Cl. A61B 1/018; A61B 1/00; A61B 1/01; A61B 8/00

(52) U.S. Cl. CPC A61B 1/018; A61B 1/00098; A61B 1/0014; A61B 1/01; A61B 8/4444

(57) ABSTRACT

An attachable endoscope assembly is disclosed herein. The attachable endoscope assembly can include an attachment component, an ultrasound assembly housing configured to selectively couple an ultrasound array to the attachable endoscope assembly, and a neck coupling the attachment component to the ultrasound assembly housing. The neck can include a guide configured to receive an instrument as it traverses out of an lumen of the endoscope and direct the instrument into a field-of-view of the ultrasound array when coupled to the ultrasound assembly housing, and a first pivot point including a first modulus of elasticity configured to move the ultrasound assembly housing between a relaxed position and a stressed position in response to a resultant force applied by the instrument when the instrument is received by the guide.

### CROSS-REFERENCE TO RELATED APPLICATIONS

The present application claims the benefit of and priority under 35 U.S.C. § 120 to U.S. Provisional Patent Application No. 63/484,556 , titled DEVICES, SYSTEMS, AND METHODS FOR GUIDING AN ENDOSCOPE INSTRUMENT AND SELECTIVELY MOVING AN ATTACHABLE ULTRASOUND ASSEMBLY, filed Feb. 13, 2023, the disclosure of which is incorporated by reference in its entirety herein.

### FIELD

The present disclosure is generally related to attachable ultrasound assemblies and, more particularly, is directed to attachable ultrasound assemblies that can particularly guide an instrument into the view of an ultrasonic transducer array and move the ultrasound transducer array to visualize a particular field of view of its surrounding environment.

### SUMMARY

The following summary is provided to facilitate an understanding of some of the innovative features unique to the aspects disclosed herein and is not intended to be a full description. A full appreciation of the various aspects can be gained by taking the entire specification, claims, and abstract as a whole.

In various aspects, an attachable endoscope assembly is disclosed. The attachable endoscope assembly can include an attachment component, an ultrasound assembly housing configured to selectively couple an ultrasound array to the attachable endoscope assembly, and a neck coupling the attachment component to the ultrasound assembly housing. The neck can include a guide configured to receive an instrument as it traverses out of an lumen of the endoscope and direct the instrument into the field-of-view of the ultrasound array when coupled to the ultrasound assembly housing, and a first pivot point including a first modulus of elasticity configured to move the ultrasound assembly housing between a relaxed position and a stressed position in response to a resultant force applied by the instrument when the instrument is received by the guide.

In other aspects, another attachable endoscope assembly is disclosed. The attachable endoscope assembly can include an attachment component configured to selectively secure the attachable endoscope assembly to an endoscope, an ultrasound assembly housing configured to selectively couple an ultrasound array to the attachable endoscope assembly, and a neck coupling the attachment component to the ultrasound assembly housing, wherein the neck includes a guide configured to receive an instrument as it traverses out of a lumen of the endoscope and direct the instrument into a field-of-view of the ultrasound array when coupled to the ultrasound assembly housing, and a first pivot point including a mechanical joint configured to move the ultrasound assembly housing between a relaxed position and a stressed position in response to a resultant force applied by the instrument when the instrument is received by the guide.

In other aspects, another attachable endoscope assembly is disclosed. The attachable endoscope assembly can include an attachment component configured to selectively secure the attachable endoscope assembly to an endoscope, an ultrasound assembly housing configured to selectively couple an ultrasound array to the attachable endoscope assembly, and a neck coupling the attachment component to the ultrasound assembly housing, wherein the neck includes a guide configured to receive an instrument as it traverses out of a lumen of the endoscope and direct the instrument into a field-of-view of the ultrasound array when coupled to the ultrasound assembly housing, a first pivot point including a first modulus of elasticity, and a second pivot point including a second modulus of elasticity wherein the first pivot point and the second pivot point are collectively configured to move the ultrasound assembly housing between a relaxed position and a stressed position in response to a resultant force applied by the instrument when the instrument is received by the guide.

These and other features and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Various features of the aspects described herein are set forth with particularity in the appended claims. The various aspects, however, both as to organization and methods of operation, together with advantages thereof, may be understood in accordance with the following description taken in conjunction with the accompanying drawings as follows:

FIG. 1 illustrates a perspective view of an assembly configured to guide an endoscope instrument and selectively move an attachable ultrasound assembly, in accordance with at least one non-limiting aspect of the present disclosure;

FIG. 2 illustrates a side view of the assembly of FIG. 1, in accordance with at least one non-limiting aspect of the present disclosure;

FIG. 3 illustrates a sectioned-side view of the assembly of FIGS. 1 and 2, with an ultrasound array installed within the attachable ultrasound assembly, in accordance with at least one non-limiting aspect of the present disclosure;

FIGS. 4A-D illustrate a side view of several other assemblies configured to guide an endoscope instrument and selectively move an attachable ultrasound assembly, in accordance with several non-limiting aspects of the present disclosure; and

FIG. 5 illustrates a side view of another assembly configured to guide an endoscope instrument and selectively move an attachable ultrasound assembly, in accordance with several non-limiting aspects of the present disclosure.

Corresponding reference characters indicate corresponding parts throughout the several views. The exemplifications set out herein illustrate various aspects of the invention, in one form, and such exemplifications are not to be construed as limiting the scope of the invention in any manner.

### DETAILED DESCRIPTION

Numerous specific details are set forth to provide a thorough understanding of the overall structure, function, manufacture, and use of the aspects as described in the disclosure and illustrated in the accompanying drawings. Well-known operations, components, and elements have not been described in detail so as not to obscure the aspects described in the specification. The reader will understand that the aspects described and illustrated herein are non-limiting examples, and thus it can be appreciated that the specific structural and functional details disclosed herein may be representative and illustrative. Variations and changes thereto may be made without departing from the scope of the claims. Furthermore, it is to be understood that such terms as “forward”, “rearward”, “left”, “right”, “upwardly”, “downwardly”, and the like are words of convenience and are not to be construed as limiting terms.

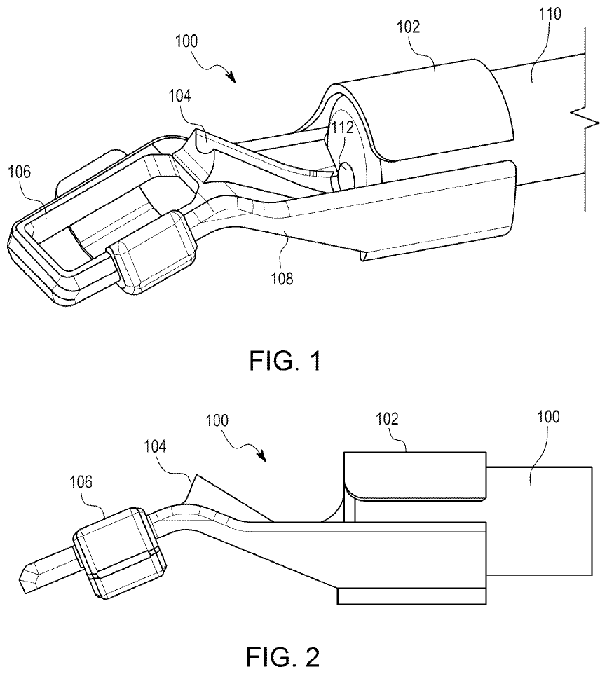

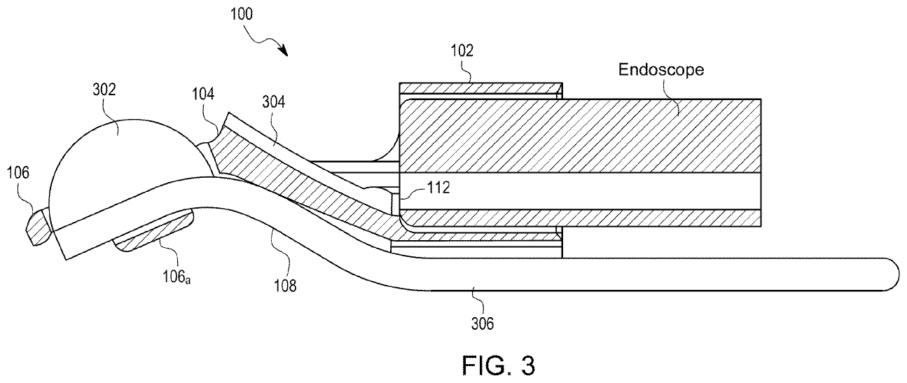

Referring now to FIG. 1, a side view of an assembly 100 configured to guide an endoscope instrument and selectively move an attachable ultrasound assembly is depicted in accordance with at least one non-limiting aspect of the present disclosure. According to the non-limiting aspect of FIG. 1, the assembly 100 can include an attachment component 102, such as a clip, configured to selectively couple the assembly 100 to an endoscope 110. Additionally, the assembly 100 can include an ultrasound transducer array housing 106 configured to selectively secure the assembly 100 to an ultrasound array 302 (FIG. 3). As depicted in FIG. 1, the assembly 100 can further include a guide 104, or ramp, configured to guide an instrument, such as a needle, that traverses through and out of a lumen 112 of the endoscope 110. According to the non-limiting aspect of FIG. 1, the guide 104 can be positioned on at least a portion of a neck 108 of the attachable ultrasound assembly 100.

Referring now to FIG. 2, a side view of the assembly 100 of FIG. 1 is depicted in accordance with at least one non-limiting aspect of the present disclosure. Here, the general structure of the guide 104 is illustrated relative to the housing 106 and endoscope 100. As such, it shall be appreciated that, as an instrument, such as a needle, traverses out of the endoscope 100 through the lumen 112 (FIG. 1), the guide will direct the needle into a particular position relative to the housing 106. For example, when the ultrasound array 302 (FIG. 3) is installed within the housing 106, the guide 104 may ensure that the needle is positioned within a field of view of the ultrasound array 302 (FIG. 3).

Referring now to FIG. 3, a sectioned side view of the assembly 100 of FIGS. 1 and 2 is depicted in accordance with at least one non-limiting aspect of the present disclosure. According to the non-limiting aspect of FIG. 3, an ultrasound array 302 has been installed into the housing 106 of the attachable ultrasound assembly 100. As such, a communications cable 306 configured to communicably couple the ultrasound array 302 to a processor is depicted as running along an outer diameter of the endoscope 100. However, according to other non-limiting aspects, the ultrasound array 302 can be communicably coupled to a processor via any means, including via a wireless network (e.g., infrastructure networks, ad hoc networks, etc.). Additionally, a sidewall 304 of the guide 104 is depicted in FIG. 3, which can prevent an instrument, or needle, of the endoscope 100 from diverting laterally, thereby preventing it from properly entering within a field-of-view of the ultrasound array 302. Instead, it shall be appreciated how the needle will traverse an upper surface of the guide 104 until it enters the field-of-view of the ultrasound array 302. The assembly 100 can further include one or more features 106ₐ configured to retain the ultrasound array 302 within the housing 106 such that it will not inadvertently decouple from the assembly 100 while in use. For example, the one or more features 106ₐ can include a a snap-fit mechanism comprising a small protrusion, or a male component (e.g., a hook, a bead, a bump, etc.) configured to deflect during assembly to catch in a female component (e.g., a hole, a depression, etc.) on the male component, such that the ultrasound array 302 can be securely retained within the housing 106. Alternately the housing 106 can have a material composition and/or be dimensioned to establish a friction fit such that the ultrasound array 302 can be securely retained within the housing 106.

Moreover, it shall be appreciated that at least a portion of the neck 108 of the assembly 100 can be particularly configured with a desired modulus of elasticity, such that a user may control an orientation of the ultrasound array 306 relative to the endoscope 100 via an input applied via the instrument or needle. For example, according to some non-limiting aspects, at least a portion of the neck 108 of the assembly 100 can include a modulus of elasticity greater than or equal to two gigapascals but less than or equal to ten gigapascals, which is particularly configured such that an average expected force applied to the at least a portion of the neck 108 will reposition the housing 106 and thus, the ultrasound array 302, through a desired, predetermined range of motions.

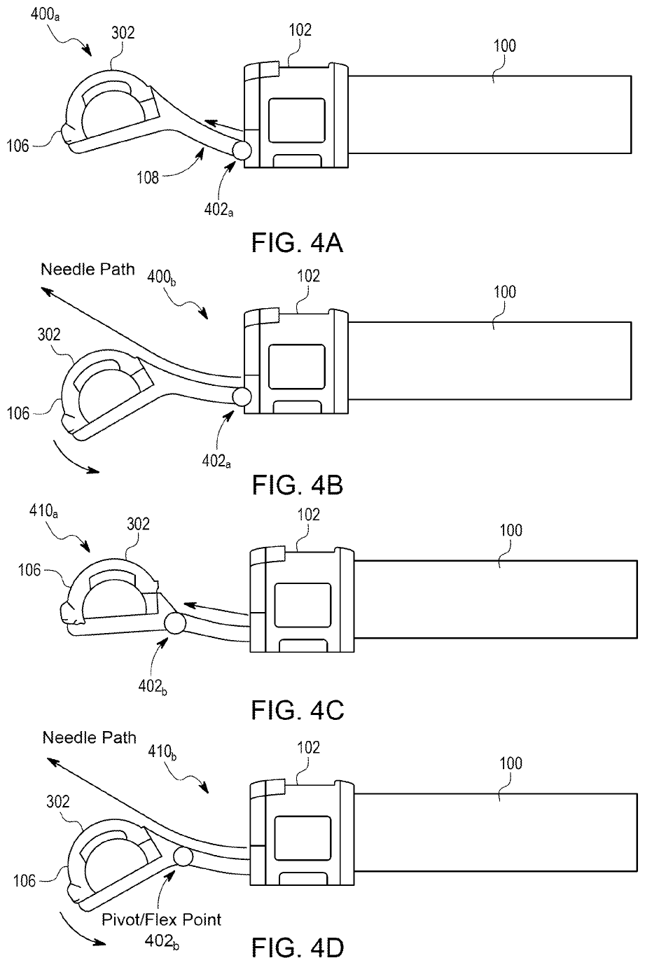

For example, referring now to FIGS. 4A-D, several side views of several other assemblies 400, 410 configured to guide an endoscope instrument and selectively move an ultrasound transducer assembly 302 are depicted in accordance with several non-limiting aspects of the present disclosure. According to the non-limiting aspects of FIGS. 4A-D, the attachable assemblies 400, 410 can be configured in a substantially similar way the attachable assembly 100 of FIGS. 1-3. However, according to the non-limiting aspects of FIGS. 4A-D, the attachable assemblies 400, 410 can be configured with intentional pivot points 402ₐ, 402_{b}, about which the neck 108 of the attachable assemblies 400, 410 are configured rotate when a force applied by a user via an instrument is applied to a guide 104. For example, according to some non-limiting aspects, the pivot points 402ₐ, 402_{b} can include a modulus of elasticity greater than or equal to two gigapascals but less than or equal to ten gigapascals. Accordingly, as an instrument, such as a needle, traverses out of a lumen 112 (FIG. 1) and into a guide, such as the guide 106 of FIGS. 1-3, the instrument will apply no less than an average expected force on the neck 108. In response to this force, the pivot points 402ₐ, 402_{b} will buckle and thus, the neck 108 will reposition the housing 106 and thus, the ultrasound array 302, through a desired, predetermined range of motions.

For example, according to the non-limiting aspect of FIGS. 4A and 4B, an attachable assembly 400 can include a pivot point 402ₐ positioned at a proximal portion of the neck 108, closer to the attachment component 102. In FIG. 4A, the instrument remains in the lumen 112 (FIG. 1) and therefore, the neck 108 remains in a relaxed, neutral position relative to the endoscope 100. However, as depicted in FIG. 4B, the instrument is traversing through and out of the lumen 112 (FIG. 1), applying a force on the neck 108 and therefore, causing the neck 108 to rotate about the pivot point 402ₐ and into a maximum stressed position when the instrument is fully extended. It shall be appreciated that, upon withdrawing the instrument back into the lumen 112 (FIG. 1) of the endoscope 100, the modulus of elasticity of the pivot point 402ₐ will cause the neck 108 to return to the relaxed, neutral position relative to the endoscope 100, as depicted in FIG. 4A. Likewise, it shall be appreciated that, as the instrument partially traverses the guide 106 (FIGS. 1-3), the neck 108 will move between any number of positions between the relaxed, neutral position of FIG. 4A and the maximum stressed position of FIG. 4B. In other words, the housing 106 and thus, the ultrasound array 302, can be selectively positioned relative to the endoscope 100 by the user, in response to specific user inputs provide via the instrument.

According to the non-limiting aspect of FIGS. 4C and 4D, an attachable assembly 400 can include a pivot point 402, positioned at a more distal portion of the neck 108, closer to the housing 106. In FIG. 4C, the instrument remains in the lumen 112 (FIG. 1) and therefore, the housing 106 remains in a relaxed, neutral position relative to the endoscope 100. This may be beneficial because, according to the non-limiting aspects of FIGS. 4C and 4D, the neck 108—and more specifically, the guide 104 (FIGS. 1-3)—can include a higher modulus of elasticity than the pivot point 402_{b} and thus, will deform less and provide more support to the instrument until the instrument arrives at the pivot point 402_{b}. At pivot point 402_{b}, deformations are preferable and necessary to reposition the housing 106 and ultrasound array 302.

According to the non-limiting aspect of FIG. 4D, the instrument is traversing through and out of the lumen 112 (FIG. 1) and has traversed a majority of the neck 108 and guide 106 (FIGS. 1-3), arriving at the pivot point 402_{b}, at which point the modulus of elasticity responds to the force applied by the instrument begins to rotate about the pivot point 402_{b}. Thus, FIG. 4B depicts the housing 106 in a maximum stressed position when the instrument is fully extended. It shall be appreciated that, upon withdrawing the instrument back into the lumen 112 (FIG. 1) of the endoscope 100, the modulus of elasticity of the pivot point 402_{b} will cause the housing 106 to return to the relaxed, neutral position relative to the endoscope 100, as depicted in FIG. 4C. Likewise, it shall be appreciated that, as the instrument partially traverses the guide 106 (FIGS. 1-3), the housing 106 will move between any number of positions between the relaxed, neutral position of FIG. 4C and the maximum stressed position of FIG. 4D. In other words, the housing 106 and thus, the ultrasound array 302, can be selectively positioned relative to the endoscope 100 by the user, in response to specific user inputs provide via the instrument.

It shall be appreciated that, aside from controlling a range of motion of the needle, the modulus of elasticity (e.g., the stiffness) of the neck 108 and/or pivot points 402ₐ, 402_{b} can also affect the range of the motion of the instrument, as well. For example, depending on the modulus of elasticity (e.g., the stiffness) of the neck 108 and/or pivot points 402ₐ, 402_{b}, the ultimate angle of instrument deflection can be either increased or reduced. For example, according to some non-limiting aspects, the modulus of elasticity can be lower, resulting in a “softer” version of the aspects of FIGS. 4A-D that may hardly cause the instrument to deflect. Alternatively, the modulus of elasticity can be higher, resulting in a “stiffer” version of the aspects of FIGS. 4A-D that may cause the instrument to deflect more than it otherwise would if it never interacted with a guide 106 (FIG. 1) of the neck 108. In this way, the amount of deflection of the instrument, or needle, and the neck 108 and/or housing 106 can range from zero instrument deflection & max subassembly deflection to max instrument deflection & zero (or at least, minimal) subassembly deflection.

According to other non-limiting aspects, two or more pivot points can be configured into the same neck of the same attachable endoscope assembly. For example, according to one non-limiting embodiment, the first pivot point 402ₐ of FIGS. 4A and 4B can be used in conjunction with the second pivot point 402ₐ of FIGS. 4C and 4D. According to such an embodiment, the second pivot point 402_{b} can be proximally positioned relative to the first pivot point 402ₐ, closer to the junction of the neck 108 and a base of the attachment endoscope assembly 102. Thus, the first pivot point 402ₐ can be configured to move the broader neck 108 assembly. The first pivot point 402ₐ, contrarily, can be distally positioned relative to the second pivot point 402ₐ, closer to the junction of the neck 108 and the ultrasound transducer array housing 106. Thus, the second pivot point 402_{b} can be configured to move the ultrasound transducer array housing 106 relative to the broader neck 108 assembly. Furthermore, since each of the first pivot point 402ₐ and the second pivot point 402ₐ can have it's own modulus of elasticity, each can be configured to produce a different (e.g., more precise, etc.) deflection, range of motion, or resistance to motion for different portions of the attachment assembly 102 (e.g., neck 108, ultrasound array 302, etc.). Such differing motions can be based on the same user input provided via the instrument.

According to still other non-limiting aspects, either of the pivot points 402ₐ, 402_{b} can include a mechanical joint and/or a spring in lieu of a particularly configured modulus of elasticity to facilitate deflection of the instrument and/or ultrasound array 302 based on user inputs provided via the instrument. Therefore, the flexibility does not necessarily have to be inherent to the design and/or geometry of the assemblies 400, 410. Regardless, it shall be appreciated that, at least the housing 106 and ultrasound array 302 can be moved as a result of the force of the instrument as it contacts the pivot points 402ₐ, 402_{b} while exiting the endoscope. The 402ₐ, 402_{b} of FIGS. 4A-D can be movably attached at one or more locations anywhere along the neck 108 of the assemblies 400, 410.

It shall be further appreciated that, according to some non-limiting aspects, it may be preferable for the assemblies 100 (FIG. 1), 400, 410 to maintain a relatively low profile, extending no more than a quarter of the diameter of the endoscope 110 beyond an outer perimeter of the endoscope 110, itself. This can enable the assemblies 100 (FIG. 1), 400, 410 to attach to a single side of the endoscope 100 while still resisting the resultant forces caused by the instrument deflection. Alternately, the present disclosure contemplates other means of attaching the assemblies 100 (FIG. 1), 400, 410 to the endoscope 100.

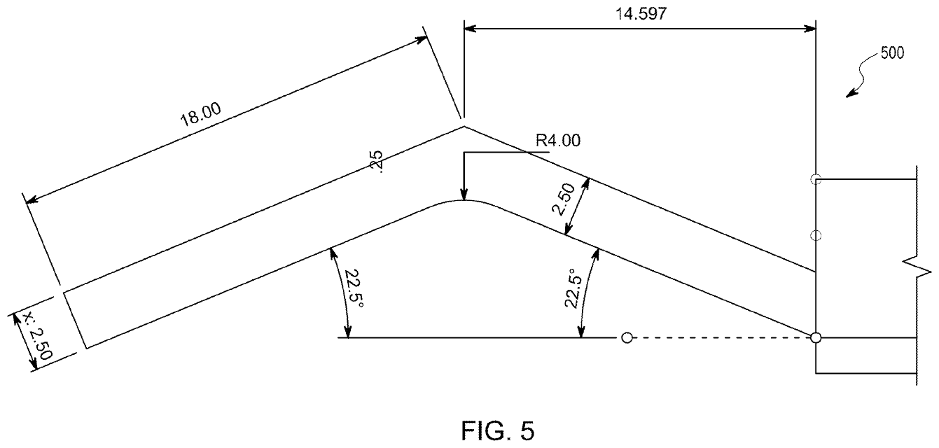

Referring now to FIG. 5, a side view of another assembly 500 configured to guide an endoscope needle and selectively move an attachable ultrasound assembly is depicted in accordance with at least one non-limiting aspect of he present disclosure. For example, the assembly 500 of FIG. 5 depicts several dimensional configurations that could be beneficially applied to the other assemblies 100 (FIG. 1), 400 (FIGS. 4A and 4B), 410 (FIGS. 4C and 4D) disclosed herein in order to facilitate the aforementioned functionality and features. However, it shall be appreciated that the non-limiting aspect of FIG. 5 is merely illustrative and that, according to other non-limiting aspects, the assemblies disclosed herein can be geometrically configured to any one of a number of preferable dimensions, based on user preference and/or intended application.

Since the inventive principles of this patent disclosure can be modified in arrangement and detail without departing from the inventive concepts, such changes and modifications are considered to fall within the scope of the following claims. The use of terms such as first and second are for purposes of differentiating different components and do not necessarily imply the presence of more than one component.

Various aspects of the subject matter described herein are set out in the following numbered clauses:

Clause 1. An attachable endoscope assembly, including: an attachment component configured to selectively secure the attachable endoscope assembly to an endoscope; an ultrasound assembly housing configured to selectively couple an ultrasound array to the attachable endoscope assembly; and a neck coupling the attachment component to the ultrasound assembly housing, wherein the neck includes: a guide configured to receive an instrument as it traverses out of a lumen of the endoscope and direct the instrument into a field-of-view of the ultrasound array when coupled to the ultrasound assembly housing; and a first pivot point including a first modulus of elasticity configured to move the ultrasound assembly housing between a relaxed position and a stressed position in response to a resultant force applied by the instrument when the instrument is received by the guide.

Clause 2. The attachable endoscope assembly according to clause 1, wherein the first modulus of elasticity is greater than or equal to two gigapascals and less than or equal to ten gigapascals.

Clause 3. The attachable endoscope according to either of clauses 1 or 2, wherein the guide includes a ramp, wherein a first end of the ramp is positioned below an opening of a lumen of the endoscope and inclines up to a second end, and wherein the second end of the ramp is positioned above the ultrasound assembly housing.

Clause 4. The attachable endoscope assembly according to any of clauses 1-3, wherein the neck further includes a second pivot point, and wherein the first pivot point and the second pivot point are collectively configured to move the ultrasound assembly housing between the relaxed position and the stressed position in response to the resultant force applied by the instrument when the instrument is received by the guide.

Clause 5. The attachable endoscope assembly according to any of clauses 1-4, wherein the second pivot point is positioned proximal to the first pivot point and includes a second modulus of elasticity, wherein the first modulus of elasticity is different than the second modulus of elasticity.

Clause 6. The attachable endoscope assembly according to any of clauses 1-5, wherein the difference between the first modulus of elasticity and the second modulus of elasticity is configured to enable the second pivot point to provide a different motion relative to the first pivot point.

Clause 7. The attachable endoscope assembly according to any of clauses 1-6, wherein the attachment component is a clip.

Clause 8. The attachable endoscope assembly according to any of clauses 1-7, wherein the guide includes a sidewall to prevent the instrument from diverting laterally.

Clause 9. The attachable endoscope assembly according to any of clauses 1-8, wherein the ultrasound assembly housing further includes an ultrasound attachment component configured to retain the ultrasound array within the housing.

Clause 10. The attachable endoscope assembly according to any of clauses 1-9, wherein the ultrasound attachment component includes a snap-fit mechanism.

Clause 11. The attachable endoscope assembly according to any of clauses 1-10, wherein the snap-fit mechanism includes a male portion configured to selectively engages a female component upon deflection, such that the ultrasound array can be retained within the ultrasound assembly housing.

Clause 12. The attachable endoscope assembly according to any of clauses 1-11, wherein the ultrasound attachment component includes a friction fit such that the ultrasound array can be securely retained within the ultrasound assembly housing.

Clause 13. The attachable endoscope assembly according to any of clauses 1-12, wherein the first pivot point is positioned at a proximal end of the neck closest to the attachment component.

Clause 14. The attachable endoscope assembly according to any of clauses 1-13, wherein the first pivot point is positioned at a distal end of the neck closest to the ultrasound assembly housing.

Clause 15. The attachable endoscope assembly according to any of clauses 1-14, wherein the attachable endoscope assembly extends no more than a quarter of a diameter of the endoscope beyond an outer perimeter of the endoscope.

Clause 16. An attachable endoscope assembly, including an attachment component configured to selectively secure the attachable endoscope assembly to an endoscope, an ultrasound assembly housing configured to selectively couple an ultrasound array to the attachable endoscope assembly, and a neck coupling the attachment component to the ultrasound assembly housing, wherein the neck includes a guide configured to receive an instrument as it traverses out of a lumen of the endoscope and direct the instrument into a field-of-view of the ultrasound array when coupled to the ultrasound assembly housing, and a first pivot point including a mechanical joint configured to move the ultrasound assembly housing between a relaxed position and a stressed position in response to a resultant force applied by the instrument when the instrument is received by the guide.

Clause 17. The attachable endoscope assembly according to clause 16, wherein the first pivot point further includes a spring attachment coupled to the mechanical joint.

Clause 18. An attachable endoscope assembly, including an attachment component configured to selectively secure the attachable endoscope assembly to an endoscope, an ultrasound assembly housing configured to selectively couple an ultrasound array to the attachable endoscope assembly, and a neck coupling the attachment component to the ultrasound assembly housing, wherein the neck includes a guide configured to receive an instrument as it traverses out of a lumen of the endoscope and direct the instrument into a field-of-view of the ultrasound array when coupled to the ultrasound assembly housing, a first pivot point including a first modulus of elasticity, and a second pivot point including a second modulus of elasticity wherein the first pivot point and the second pivot point are collectively configured to move the ultrasound assembly housing between a relaxed position and a stressed position in response to a resultant force applied by the instrument when the instrument is received by the guide.

Clause 19. The attachable endoscope assembly according to clause 18, wherein the first modulus of elasticity is greater than or equal to two gigapascals and less than or equal to ten gigapascals.

Clause 20. The attachable endoscope assembly according to either of clauses 18 or 19, wherein the guide includes a ramp, wherein a first end of the ramp is positioned below an opening of a lumen of the endoscope and inclines up to a second end, and wherein the second end of the ramp is positioned above the ultrasound assembly housing.

All patents, patent applications, publications, or other disclosure material mentioned herein, are hereby incorporated by reference in their entirety as if each individual reference was expressly incorporated by reference respectively. All references, and any material, or portion thereof, that are said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference and the disclosure expressly set forth in the present application controls.

The present invention has been described with reference to various exemplary and illustrative aspects. The aspects described herein are understood as providing illustrative features of varying detail of various aspects of the disclosed invention; and therefore, unless otherwise specified, it is to be understood that, to the extent possible, one or more features, elements, components, constituents, ingredients, structures, modules, and/or aspects of the disclosed aspects may be combined, separated, interchanged, and/or rearranged with or relative to one or more other features, elements, components, constituents, ingredients, structures, modules, and/or aspects of the disclosed aspects without departing from the scope of the disclosed invention. Accordingly, it will be recognized by persons having ordinary skill in the art that various substitutions, modifications or combinations of any of the exemplary aspects may be made without departing from the scope of the invention. In addition, persons skilled in the art will recognize, or be able to ascertain using no more than routine experimentation, many equivalents to the various aspects of the invention described herein upon review of this specification. Thus, the invention is not limited by the description of the various aspects, but rather by the claims.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although claim recitations are presented in a sequence(s), it should be understood that the various operations may be performed in other orders than those which are described, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Directional phrases used herein, such as, for example and without limitation, top, bottom, left, right, lower, upper, front, back, and variations thereof, shall relate to the orientation of the elements shown in the accompanying drawing and are not limiting upon the claims unless otherwise expressly stated.

The terms “about” or “approximately” as used in the present disclosure, unless otherwise specified, means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 200%, 105%, 100%, 9%, 8%, 7%, 6%, 5%, 4%, 3%, 2%, 1%, 0.5%, or 0.05% of a given value or range.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 100” includes all sub-ranges between (and including) the recited minimum value of 1 and the recited maximum value of 100, that is, having a minimum value equal to or greater than 1 and a maximum value equal to or less than 100. Also, all ranges recited herein are inclusive of the end points of the recited ranges. For example, a range of “1 to 100” includes the end points 1 and 100. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, any reference to a processor or microprocessor can be substituted for any “control circuit,” which may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

1. An attachable endoscope assembly, comprising:
an attachment component configured to selectively secure the attachable endoscope assembly to an endoscope;
an ultrasound assembly housing configured to selectively couple an ultrasound array to the attachable endoscope assembly; and
a neck coupling the attachment component to the ultrasound assembly housing, wherein the neck comprises:
a guide configured to receive an instrument as it traverses out of a lumen of the endoscope and direct the instrument into a field-of-view of the ultrasound array when coupled to the ultrasound assembly housing; and
a first pivot point comprising a first modulus of elasticity configured to move the ultrasound assembly housing between a relaxed position and a stressed position in response to a resultant force applied by the instrument when the instrument is received by the guide, wherein the first modulus of elasticity is greater than or equal to two gigapascals and less than or equal to ten gigapascals.

2. (canceled)

3. The attachable endoscope assembly of claim 1, wherein the guide comprises a ramp, wherein a first end of the ramp is positioned below an opening of a lumen of the endoscope and inclines up to a second end, and wherein the second end of the ramp is positioned above the ultrasound assembly housing.

4. The attachable endoscope assembly of claim 1, wherein the neck further comprises a second pivot point, and wherein the first pivot point and the second pivot point are collectively configured to move the ultrasound assembly housing between the relaxed position and the stressed position in response to the resultant force applied by the instrument when the instrument is received by the guide.

5. The attachable endoscope assembly of claim 4, wherein the second pivot point is positioned proximal to the first pivot point and comprises a second modulus of elasticity, wherein the first modulus of elasticity is different than the second modulus of elasticity.

6. The attachable endoscope assembly of claim 5, wherein the difference between the first modulus of elasticity and the second modulus of elasticity and enables the second pivot point to move differently relative to the first pivot point.

7. The attachable endoscope assembly of claim 1, wherein the attachment component is a clip.

8. The attachable endoscope assembly of claim 1, wherein the guide comprises a sidewall to prevent the instrument from diverting laterally.

9. The attachable endoscope assembly of claim 1, wherein the ultrasound assembly housing further comprises an ultrasound attachment component configured to retain the ultrasound array within the housing.

10. The attachable endoscope assembly of claim 9, wherein the ultrasound attachment component comprises a snap-fit mechanism.

11. The attachable endoscope assembly of claim 10, wherein the snap-fit mechanism comprises a male portion configured to selectively engage a female component upon deflection, such that the ultrasound array can be retained within the ultrasound assembly housing.

12. The attachable endoscope assembly of claim 9, wherein the ultrasound attachment component comprises a friction fit such that the ultrasound array can be securely retained within the ultrasound assembly housing.

13-17. (canceled)

18. An attachable endoscope assembly, comprising:
an attachment component configured to selectively secure the attachable endoscope assembly to an endoscope;
an ultrasound assembly housing configured to selectively couple an ultrasound array to the attachable endoscope assembly; and
a neck coupling the attachment component to the ultrasound assembly housing, wherein the neck comprises:
a guide configured to receive an instrument as it traverses out of a lumen of the endoscope and direct the instrument into a field-of-view of the ultrasound array when coupled to the ultrasound assembly housing;
a first pivot point comprising a first modulus of elasticity; and
a second pivot point comprising a second modulus of elasticity wherein the first pivot point and the second pivot point are collectively configured to move the ultrasound assembly housing between a relaxed position and a stressed position in response to a resultant force applied by the instrument when the instrument is received by the guide.

19. The attachable endoscope assembly of claim 18, wherein the first modulus of elasticity is greater than or equal to two gigapascals and less than or equal to ten gigapascals.

20. The attachable endoscope assembly of claim 19, wherein the guide comprises a ramp, wherein a first end of the ramp is positioned below an opening of a lumen of the endoscope and inclines up to a second end, and wherein the second end of the ramp is positioned above the ultrasound assembly housing.

21. An attachable endoscope assembly, comprising:
an attachment component configured to selectively secure the attachable endoscope assembly to an endoscope;
an ultrasound assembly housing configured to selectively couple an ultrasound array to the attachable endoscope assembly; and
a neck coupling the attachment component to the ultrasound assembly housing, wherein the neck comprises:
a guide configured to receive an instrument as it traverses out of a lumen of the endoscope and direct the instrument into a field-of-view of the ultrasound array when coupled to the ultrasound assembly housing; and
a first pivot point comprising a first modulus of elasticity configured to move the ultrasound assembly housing between a relaxed position and a stressed position in response to a resultant force applied by the instrument when the instrument is received by the guide, wherein the neck further comprises a second pivot point, and wherein the first pivot point and the second pivot point are collectively configured to move the ultrasound assembly housing between the relaxed position and the stressed position in response to the resultant force applied by the instrument when the instrument is received by the guide, wherein the second pivot point is positioned proximal to the first pivot point and comprises a second modulus of elasticity, wherein the first modulus of elasticity is different than the second modulus of elasticity.

22. An attachable endoscope assembly, comprising:
an attachment component configured to selectively secure the attachable endoscope assembly to an endoscope;
an ultrasound assembly housing configured to selectively couple an ultrasound array to the attachable endoscope assembly; and
a neck coupling the attachment component to the ultrasound assembly housing, wherein the neck comprises:
a guide configured to receive an instrument as it traverses out of a lumen of the endoscope and direct the instrument into a field-of-view of the ultrasound array when coupled to the ultrasound assembly housing; and
a first pivot point comprising a first modulus of elasticity configured to move the ultrasound assembly housing between a relaxed position and a stressed position in response to a resultant force applied by the instrument when the instrument is received by the guide, wherein the second pivot point is positioned proximal to the first pivot point and comprises a second modulus of elasticity, wherein the ultrasound assembly housing further comprises an ultrasound attachment component configured to retain the ultrasound array within the housing.
